(19) United States
(12) Patent Application Publication
(10) Pub. No.: US 2006/0084915 A1
(43) Pub. Date: Apr. 20, 2006

(54) Hub locking structure for safety syringe

(76) Inventor: Jin Rong Chang, Chu-Pei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/890,157

(22) Filed: Jul. 14, 2004

(51) Int. Cl. A61M 5/00; A61M 5/32

(52) U.S. Cl. 604/110; 604/198

(57) ABSTRACT

A hub locking structure for safety syringe includes a hub-locking member that is initially located in a rear part of a hub fitted in an inner front of a barrel before the safety syringe is used to inject. In this initial position, a flange ring on the hub-locking member radially pushes the rear part of the hub outward to firmly engage an annular groove on the hub with a retaining ring on the inner wall of the barrel. When a plunger in the barrel is pushed forward to inject, the hub-locking member is moved further into the hub with the flange ring engaged with retaining holes on the hub to disengage the annular groove on the hub from the retaining ring of the barrel, and thereby allows the hub and the hub-locking member to be pulled backward by the plunger to retract a needle into the barrel.

### FIELD OF THE INVENTION

The present invention relates to a hub locking structure for safety syringe, and more particularly to a hub locking structure that ensures firm and airtight connection of a hub to a barrel of a safety syringe before the latter is used to inject, and enables smooth pull of a needle connected to the hub into the barrel after the safety syringe has been used to inject.

### BACKGROUND OF THE INVENTION

In a conventional syringe, a used needle thereof is removed from a hub and disposed separately. To prevent the used and separated needle from being used for a second time or causing any infection or environmental contamination, a safety syringe with a retractable needle has been developed. The safety syringe must be designed to ensure stable and airtight connection of the hub to the barrel and smooth pull of the used needle into the barrel. Most of the currently available safety syringes are not ideal in terms of the above-mentioned two requirements.

### SUMMARY OF THE INVENTION

A primary object of the present invention is to provide a hub locking structure for safety syringe that ensures stable and airtight connection of a hub to a barrel of the safety syringe, and smooth pull of a used needle into the barrel.

To achieve the above and other objects, the hub locking structure for safety syringe according to the present invention mainly includes a barrel being provided on an inner wall surface with a shoulder portion close to a front end of the barrel, and a radially protruded retaining ring behind the shoulder portion by a predetermined distance; a hub being fitted in the barrel immediately behind the front end of the barrel, and including a sleeve portion and a central tube portion forward projected from a front end of the sleeve portion, the tube portion defining an internal through hole to communicate with an inner space of the sleeve portion, the hub also being provided on a peripheral wall of the sleeve portion at predetermined positions with through retaining holes, and on an outer wall surface of the peripheral wall of the sleeve portion at predetermined positions with a plurality of grooves, and the sleeve portion being provided on a front inner wall surface with screw threads for a needle to screw thereto; and a hollow cylindrical hub-locking member located in the sleeve portion of the hub, and defining a central hole for a plunger to extend a front head thereinto, the central hole being provided on an outer wall surface at a predetermined position with a radially outward flange ring corresponding to the retaining holes on the hub.

When the plunger is pushed forward to force out a medical liquid from the barrel, the front head of the plunger is received in the central hole of the hub-locking member to move the hub-locking member further into the sleeve portion of the hub until the flange ring on the hub-locking member is engaged with the retaining holes on the hub; and then, when the plunger is pulled backward, the hub coupled with the hub-locking member along with a needle connected to the hub are pulled rearward into the barrel.

When the hub is fitted in the barrel, an outer wall surface of the hub is a butted at a predetermined position on the shoulder portion on the inner wall surface of the barrel, ensuring stable and airtight location of the hub in the barrel in an injecting direction.

The hub is provided on the outer wall surface with annular grooves and axial grooves to allow a rear part of the hub to expand outward and deform when the flange ring on the hub-locking member outward pushes against the hub. When the rear part of the hub is outward expanded and deformed, one of the annular grooves is firmly engaged with the retaining ring on the inner wall surface of the barrel, so that the hub is firmly clamped to the barrel.

The central hole of the hub-locking member is a stepped hole to hold a front head of the plunger in the central hole, and provided on a rear end surface with an annular groove, which is communicated with the central hole via a plurality of radial slits. The annular groove and the radial slits on the hub-locking member serve as passages for the medical liquid and provide an elasticity for the plunger to extend into the central hole.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an exploded and partially cutaway perspective view of a hub locking structure for safety syringe according to the present invention;

FIG. 2 is a partially phantom perspective view showing the relation between a hub and a hub-locking member of the safety syringe of FIG. 1;

FIG. 3 is an assembled sectional view of the hub locking structure of the present invention before the safety syringe is used to inject;

FIG. 4 shows a plunger of the safety syringe is pushed toward the hub locking structure of the present invention to effect an injection;

FIG. 5 shows the hub-locking member is fully engaged with the hub when the plunger has been fully pushed forward to complete the injection; and

FIG. 6 shows a used needle connected to the hub is pulled backward into a barrel of the safety syringe via the hub locking structure of the present invention.

### DETAILED DESCRIPTION OF THE PREFERRED EMBODIMENTS

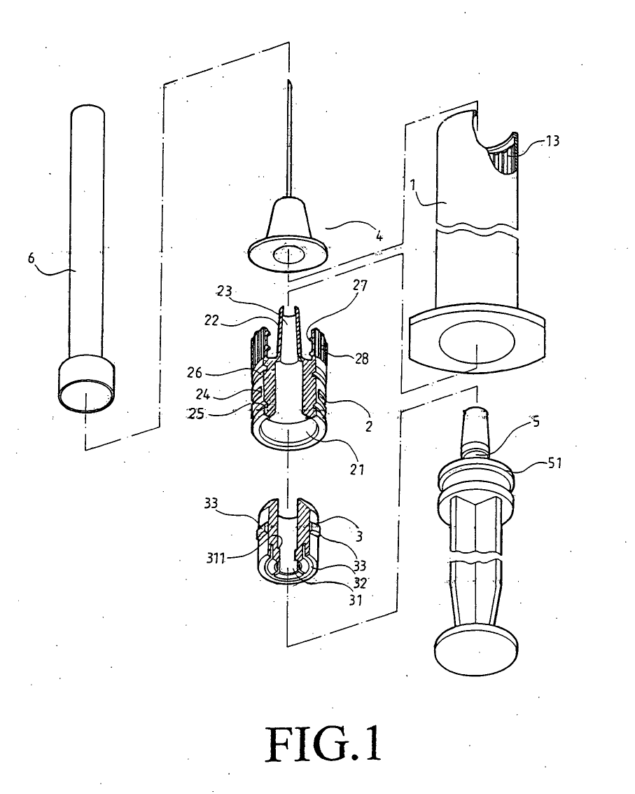

Please refer to FIGS. 1, 2, and 3 at the same time, in which a hub locking structure for safety syringe according to the present invention is shown. The safety syringe mainly includes a barrel 1, a hub 2, a hub-locking member 3, a needle 4, a plunger 5, and a cap 6. The barrel 1, the hub 2, and the hub-locking member 3 together constitute the hub locking structure of the present invention.

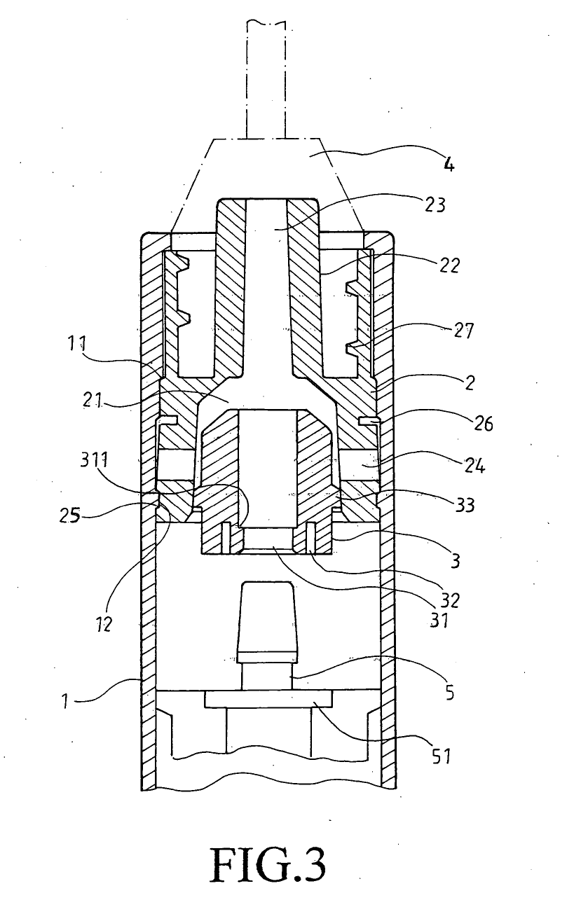

As can be most clearly seen from FIGS. 1 and 3, the barrel 1 is provided on an inner wall surface with a shoulder portion 11 close to a front end of the barrel, a radially protruded retaining ring 12 behind the shoulder portion 11 by a predetermined distance, and a plurality of parallelly arranged teeth 13 axially extended backward from the front end of the barrel by a predetermined length.

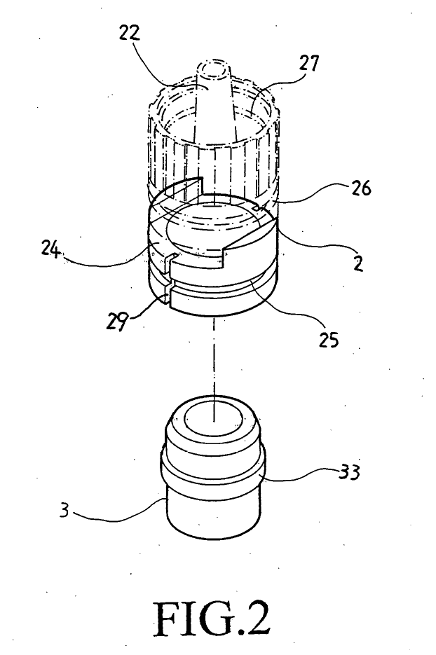

The hub 2 includes a sleeve portion 21, and a central tube portion 22 forward projected from a front end of the sleeve portion 21. The tube portion 22 defines an internal through hole 23 to communicate with an inner space of the sleeve portion 21. As can be most clearly seen from FIG. 2, the hub 2 is provided on a rear part of a peripheral wall of the sleeve portion 21 with a plurality of through retaining holes 24, and on an outer surface of the peripheral wall of the sleeve portion 21 with annular grooves 25, 26 located below and above the retaining holes 24, respectively, and opposite vertical grooves 29 corresponding to the retaining holes 24. The hub 2 is also provided on an inner wall surface of a front part of the peripheral wall of the sleeve portion 21 surrounding the tube portion 22 with screw threads 27 for the needle 4 to screw thereto, and on outer wall surface of the front part of the peripheral wall of the sleeve portion 21 with a plurality of parallelly arranged and axially extended grooves 28 corresponding to the grooves 13 on the front inner wall surface of the barrel 1. When the hub 2 is fitted in the barrel 1 with the grooves 28 engaged with the teeth 13, the hub 2 is prevented from rotating relative to the barrel 1 when the needle 4 is screwed to the hub 2.

The hub-locking member 3 is a hollow cylindrical member defining a central hole 31 for a front head of the plunger 5 to extend thereinto. The central hole 31 is provided on an inner wall surface close to a rear end thereof with a shoulder portion 311, on an outer wall surface close to a middle section with a radially outward flange ring 22 corresponding to the retaining holes 24 on the hub 2, on a rear end surface with an annular groove 32, and between the inner wall surface and the annular groove 32 with a plurality of circumferentially spaced slits.

As can be seen from FIG. 3, when the hub 2 is fitted in the barrel 1, a front end of the sleeve portion 21 is abutted against an inner side of a front opening of the barrel 1, a middle section of the sleeve portion 21 is abutted on the shoulder portion 11 on the inner wall of the barrel 1 to form a locating and airtight joint thereat, and the grooves 28 are meshed with the teeth 13. The hub-locking member 3 is initially located close to a rear part of the inner space defined by the sleeve portion 21 with the flange ring 22 outward pushing against a rear inner wall surface of the sleeve portion 21 of the hub 2. With the annular groove 26 above the retaining holes 24 and the vertical grooves 29 on the sleeve portion 21 of the hub 2, and the outward pushing force of the flange ring 22 of the hub-locking member 3 against the rear inner wall surface of the hub 2, the rear part of the sleeve portion 21 of the hub 2 is slightly radially expanded and deformed, causing the annular groove 25 below the retaining holes 24 to firmly engage with the retaining ring 12 on the inner wall surface of the barrel 1. These arrangements provide a strong clamping effect to firmly clamp the hub 2 in the barrel 1 before the safety syringe is used to inject.

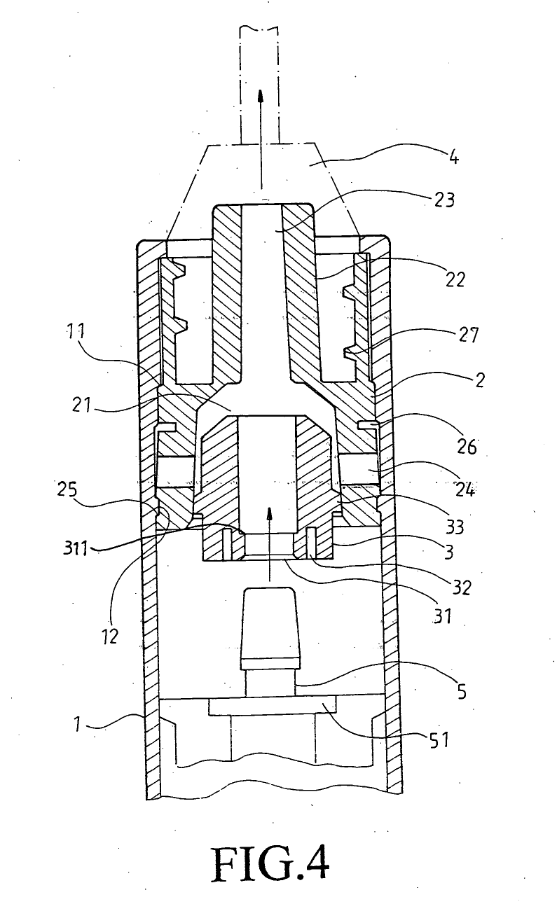

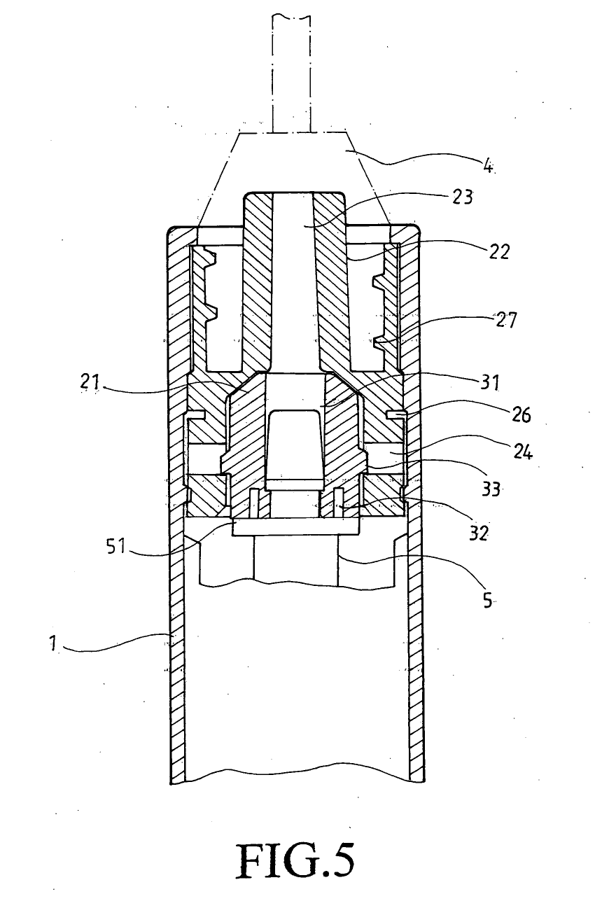

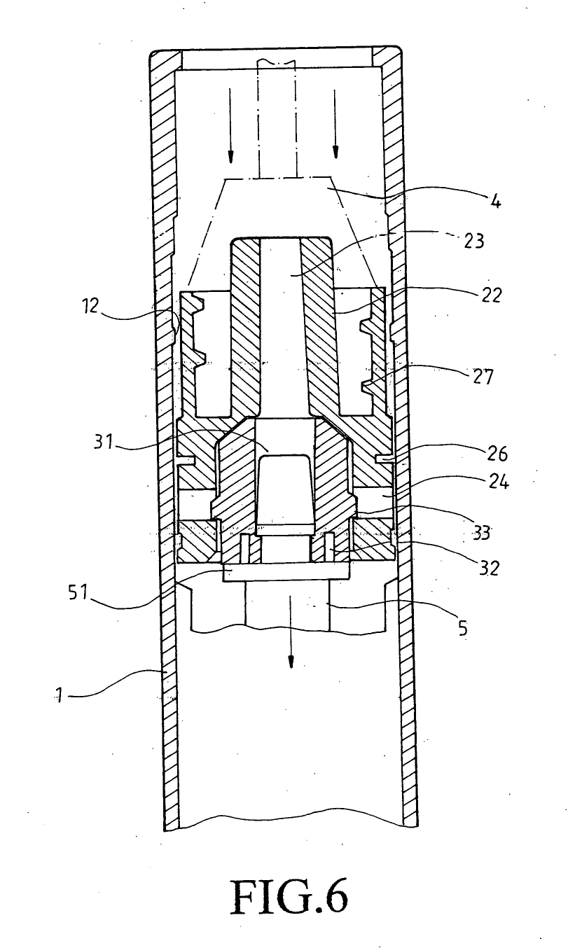

To use the safety syringe of the present invention to inject, first push the plunger 5 forward as shown in FIG. 4, so that an amount of medical liquid in the barrel 1 is forced through the tube portion 22 of the hub 2 to jet from the needle 4. When the plunger 5 is pushed forward to engage the whole front head thereof with the central hole 31 of the hub-locking member 3, a rear end of the front head is abutted on the shoulder portion 311 in the central hole 31 and prevented from moving backward, and a finger flange 51 closely behind the front head of the plunger 5 is abutted against the rear end surface of the hub-locking member 3 to move the hub-locking member 3 further into the sleeve portion 21, causing the flange ring 33 to align and thereby engage with the retaining holes 24 on the hub 2, as shown in FIG. 5. At this point, the flange ring 33 no longer outward pushes against the sleeve portion 21 of the hub 2, allowing the rear part of the sleeve portion 21 to restore from the outward expanded and deformed state, and the annular groove 25 to disengage from the retaining ring 12 of the barrel 1. With the flange ring 33 held in the retaining holes 24 and the front head of the plunger 5 in the central hole 31 of the hub-locking member 3, and the annular groove 25 disengaged from the retaining ring 12, the plunger 5 may be pulled backward to move the hub 2 and the hub-locking member 3 rearward until the whole needle 4 is retracted into the barrel 1, as shown in FIG. 6.

Please refer back to FIG. 4. To use the safety syringe of the present invention to inject, the plunger 5 is pushed forward to release the medical liquid in the barrel 1 from the needle 4 via the hub 2.

In FIG. 5, the medical liquid has been fully jetted and the front head of the plunger 5 is completely extended into the central hole 31 of the hub-locking member 3. The annular groove 32 at the rear end surface of the hub-locking member 3, and the circumferentially spaced slits between the central hole 31 and the annular groove 32 together form a buffer for the front head of the plunger 5 to move into the central hole 31 easier. Once the front head of the plunger 5 has entered the central hole 31 of the hub-locking member 3, it is held in the central hole 31 by abutting the rear end of the front head on the shoulder portion 311. The plunger 5 engaged with the central hole 31 keeps pushing the hub-locking member 3 forward into the sleeve portion 21 of the hub 2 until the flange ring 33 is engaged with the retaining holes 24 on the hub 2 to firmly hold the hub-locking member 3 to the hub 2 without moving any further. Meanwhile, the annular groove 25 on the hub 2 disengages from the retaining ring 12 on the barrel 1 to facilitate smooth pull of the needle 4 into the barrel 1.

In FIG. 6, the firmly coupled hub 2 and hub-locking member 3 are moved backward by pulling the plunger 5 rearward, so that the needle 4 is fully retracted into the barrel 1 and prevented from contaminating the external environment.

The hub 2 with the retaining holes 24 and the hub-locking member 3 with the flange ring 33 together produce a clamping effect and an airtight joint in the safety syringe and enable easy pull of the needle into the barrel to prevent undesired contamination of external environment by the needle after the safety syringe has been used to inject.

1. A hub locking structure for safety syringe, comprising:
a barrel being provided on an inner wall surface with a shoulder portion close to a front end of the barrel and a radially protruded retaining ring behind the shoulder portion by a predetermined distance, said barrel including a plurality of teeth parallel one to another and axially extending from the front end of the barrel;
a hub being fitted in said barrel immediately behind the front end of said barrel, and including a sleeve portion and a central tube portion forward projected from a front end of said sleeve portion, said tube portion defining an internal through hole to communicate with an inner space of said sleeve portions said hub also being provided on a peripheral wall of said sleeve portion at predetermined positions with a plurality of through retaining holes, said hub being further provided on a peripheral wall thereof with a plurality of axially extending grooves parallel one to another, said grooves on said hub corresponding to said teeth on said barrel and engaged therewith so as to prevent said hub from rotating relative to said barrel; and
a hub-locking member being a hollow cylindrical member located in said sleeve portion of said hub and defining a central hole for a plunger to extend a front head thereinto, said central hole being provided on an inner wall surface at a predetermined position with a shoulder portion, said hub-locking member having on an outer wall surface at a predetermined position thereof a radially outward flange ring corresponding to said retaining holes on said hub and having on a rear end surface thereof an annular groove;
whereby when said plunger is pushed forward into said barrel to effect an injection, the front head of said plunger is received in said central hole of said hub-locking member to move said hub-locking member into said sleeve portion of said hub until said flange ring on said hub-locking member is engaged with said retaining holes on said hub so that when said plunger is pulled axially away from said front end of the barrel subsequent to said injection, said hub coupled with said hub-locking member are retracted into said barrel.

2. The hub locking structure for safety syringe as claimed in claim 1, wherein said hub-locking member is initially fitted in a rear part of said sleeve portion of said hub before the safety syringe is used to inject, such that said flange ring on said hub-locking member outward pushes against said hub to expand and slightly deform the rear part of said sleeve portion and cause a rear one of said grooves on the outer wall surface of said hub to engage with said retaining ring on the inner wall surface of said barrel, and thereby provides a clamping effect to firmly hold said hub in said barrel before said safety syringe is used to inject.

3. The hub locking structure for safety syringe as claimed in claim 2, wherein when said hub-locking member is moved further into said sleeve portion of said hub by said plunger with said flange ring on said hub-locking member aligned and engaged with said retaining holes on said hub, said rear groove on the outer wall surface of said hub is disengaged from said retaining ring on the inner wall surface of said barrel and said hub is no longer clamped to said barrel.
